(19) United States
(12) Patent Application Publication
(10) Pub. No.: US 2020/0397419 A1
(43) Pub. Date: Dec. 24, 2020

(54) BIOPSY DEVICE

(71) Applicants: Chad M. Buchanan, Mequon, WI (US); James Taylor, Venice, FL (US)

(72) Inventors: Chad M. Buchanan, Mequon, WI (US); James Taylor, Venice, FL (US)

(21) Appl. No.: 16/908,258

(22) Filed: Jun. 22, 2020

Related U.S. Application Data
(60) Provisional application No. 62/865,157, filed on Jun. 22, 2019.

(51) Int. Cl. A61B 10/02

(52) U.S. Cl. CPC A61B 10/0275; A61B 2010/0208

(57) ABSTRACT

A biopsy device may include a stylet having a stylet tip and an outer slot for receiving a biopsy sample, a cannula extending about and receiving the stylet, the cannula having a cannula tip with a cutting edge facing an axial centerline of the cannula and a cannula drive coupled to the cannula to translate and rotate the cannula along and about the axial centerline of the cannula from a first position in which the outer slot is uncovered and a second position in which the cannula extends over and covers the outer slot.

### CROSS-REFERENCE TO RELATED APPLICATION

The present application is a non-provisional US patent application claiming priority under 35 USC 119 from co-pending U.S. Provisional Patent Application Ser. No. 62/865,157 filed on Jun. 22, 2019 by Buchanan et al. and entitled BIOPSY DEVICE, the full disclosure of which is hereby incorporated by reference.

### BACKGROUND

Biopsy devices are sometimes used to obtain biological samples of tissue for analysis.

### BRIEF DESCRIPTION OF THE DRAWINGS

FIG. 1 is a sectional view illustrating portions of an example biopsy device.

FIG. 2 is a sectional view of the biopsy device of FIG. 1 taken along line 2-2.

FIG. 3 is a sectional view of portions of another example biopsy device.

FIGS. 4A, 4B, 4C, 4D, 4E, 4F, 4G, 4H and 41 illustrate the example method for obtaining a biopsy sample, such as with the biopsy device of FIG. 1 or FIG. 3.

FIG. 5 is a top view of an example biopsy device

FIG. 6 is a front perspective view of the biopsy device of FIG. 5.

FIG. 7 is a side view of the biopsy device of FIG. 5.

FIG. 8 is a front view of the biopsy device of FIG. 7.

FIG. 9 is a rear view of the biopsy device of FIG. 5.

FIG. 10 is an exploded perspective view of the biopsy device of FIG. 5, omitting an example stylet and cannula.

FIG. 11A is a sectional view of the biopsy device of FIG. 5 with the stylet having an outer slot in a covered position or state.

FIG. 11B is a sectional view of the biopsy device of FIG. 5 during initial insertion into biological tissue.

FIG. 12A is a sectional view of the biopsy device of FIG. 5 with the stylet extended to uncover its outer slot.

FIG. 12B is an enlarged view of the extended stylet of FIG. 12A.

FIG. 12C is a sectional view of the biopsy device of FIG. 12A inserted into biological tissue with the outer slot position within tissue of interest.

FIG. 13 is a sectional view of the biopsy device of FIG. 5 illustrating initial translation and rotation of the cannula towards a tip of the stationary stylet.

FIG. 14 is a sectional view of the biopsy device of FIG. 5 illustrating further translation and rotation of the cannula towards the tip of the stationary stylet.

FIG. 15 is an enlarged view of the cannula and stylet of FIG. 14.

FIG. 16 is a partially transparent view of the biopsy device of FIG. 15A while inserted within the biological tissue.

FIG. 17 is a sectional view of the biopsy device of FIG. 5 illustrating further translation and rotation of the cannula towards the tip of the stationary stylet.

FIG. 18 is a sectional view of the biopsy device of FIG. 17.

FIG. 19 is a partially transparent view of the biopsy device of FIG. 19A while within the biological tissue with the cannula extending over the slot of the stylet.

FIG. 20 is a partially transparent view illustrating the biopsy device of FIG. 5 being withdrawn from the biological tissue to withdraw the biological tissue sample contained within the outer slot of the stylet.

FIG. 21 is an exploded perspective view of an example biopsy device.

FIGS. 22A, 22B, 22C and 22D are sectional views of the biopsy device of FIG. 21 during different stages of sample acquisition.

FIG. 23A is a top view of an example biopsy device.

FIG. 23B is a side view of the biopsy device of FIG. 23A.

FIG. 23C is an enlarged view of the biopsy device of FIG. 23B taken along line 23C-23C.

FIG. 23D is a sectional view of the biopsy device of FIG. 23B taken along line 23B.

FIG. 23E is an enlarged view taken along line 23E-23E of FIG. 23C.

FIG. 24 is an exploded perspective view of the biopsy device of FIG. 23A.

FIG. 25 is a sectional view of the biopsy device of FIG. 23A in an unloaded state.

FIG. 26 is a sectional view of the biopsy device of FIG. 23A in a loaded state.

FIG. 27 is a sectional view of the biopsy device of FIG. 23A upon firing of the loaded biopsy device.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION OF EXAMPLES

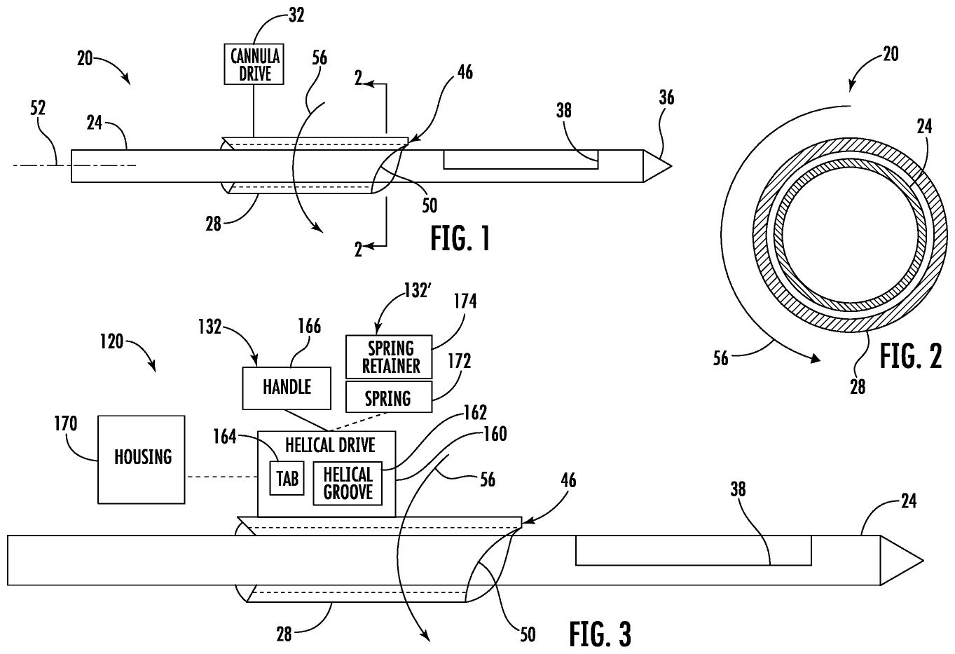

FIGS. 1 and 2 illustrate an example biopsy device 20. FIG. 1 illustrates portions of an example biopsy device 20. FIG. 2 is a sectional view of biopsy device 20 take along line 2-2. Biopsy device 20 comprises stylet 24, cannula 28 and cannula drive 32. Stylet 24 comprises an elongate hollow tube terminating at a closed ended and pointed tip or knife edge 50 and outer slot 38 for receiving a biopsy sample.

Cannula comprises a hollow elongate to extending about and receiving stylet 24. Cannula 28 itself has a cannula tip 46 with a cutting edge 50 which faces an axial centerline 52 of cannula 28. In one implementation, cutting edge 50 extends in a plane less than 45° to the axial centerline of the cannula. In one implementation, the cutting edge comprise different edge segments or portions about the axial centerline, wherein at least one of the portions extends in a plane less than 45° to the axial centerline of the cannula. Because at least portions of the cutting edge extend in a plane less than 45° to the axial centerline of the cannula, the cutting edge is more closely parallel to the axial centerline of the cannula to provide enhanced cutting or severing of biological tissue as the cannula is rotated about the axial centerline of the cannula and/or the axial centerline of the stylet. As will be described hereafter with perfect to biopsy device 320, in other implementations, the cutting edge may be a straight cut, extending in a single plane that extends 45° to the axial centerline of the cannula.

Cannula drive 32 (schematically illustrated) is operably coupled to the cannula 28 so as to at least rotate cannula 28 (as indicated by arrow 56) as cannula 28 is being translated along the axial centerline 52 of cannula 28 along the axial centerline of stylet 24 from a first position in which the outer slot 38 is uncovered (ash currently shown in 1) and a second position in which cannula 28 extends over and covers the outer slot 38. FIG. 2 is a sectional view illustrating portions of the biopsy device 20. In one implementation, cannula drive 32 rotates cannula 28 as the user manually translates or moves cannula 28 along an relative to stylet 24. In other implementations cannula drive 32 performs both the rotation and linear translation of cannula 28. In implementations where cannula Drive 30 is to perform both the rotation and linear translation of cannula 28, such rotation and linear translation are synced.

FIG. 3 illustrates portions of an example biopsy device 120. Biopsy device 120. Biopsy device 120 is similar to biopsy device 20 except that biopsy device 120 is illustrated as comprising a cannula drive 132. Cannula drive 132 comprises a helical drive 160 comprising a helical groove or thread 162 and tab 164 projecting into the helical thread or groove 162. In one implementation, tab 164 may itself comprise a helical thread intermeshing with the helical thread 162. Cannula drive 132 further comprises handle 166. One of the helical groove and the tab is directly or indirectly connected to cannula 28 so as to move and rotate with cannula 28 while the other of the helical thread or groove 162 and tab 164 is connected to handle 166. Handle 166 is slidably supported for movement along the axial centerline 52 of cannula 28 and along the axis centerline of stylet 24. Linear movement of handle 166 results in linear movement of the other of the helical thread 162 and tab 164 which results in concurrent linear translation and rotation of cannula 28.

As indicated by broken lines, in another implementation, biopsy device 120 may alternatively comprise cannula drive 132′. Cannula drive 132′ also uses helical drive 160, but comprises housing 170, Spring 172 and spring retainer 174 to rotate and translate cannula 28. Housing 170 comprises a structure about or adjacent to cannula 28. In one implementation, housing 170 (schematically shown) is configured to be grasped by user and held substantially stationary as cannula 28 is rotated and translated. Housing 170 is fixed to one of the tab 164 and the helical groove 162 of helical drive 160. Spring 172 is captured between housing 170 and the other of the helical groove 162 and tab 164 so as to urge the other of the helical groove 162 and tab 164 in a direction along an axis centerline the stylet 24 relative to said one of the helical groove 162 and tab 164 which causes cannula 28 to rotate while being linearly translated, relative to stylet 24, along the axial centerline of stylet 24.

In some implementations, different axial segments of helical groove 162 have geometries (a tighter or closer fit with respect to tab 164) or surface characteristics (a rougher surface or a coating or layer of material with a higher coefficient of friction with respect to the material of tab 164) that result in different degrees of friction and different degrees of resistance with respect to relative movement between 164 and helical groove 162. As a result, the speed at which cannula 28 rotates and moves about stylet 24 may be adjusted automatically based upon the actual positioning of cannula 28 relative to stylet 24. Cannula 28 may rotate and translate at a first nonzero speed across a first portion of stylet 24 and may rotate and translate at a second nonzero speed across a second different portion of stylet 24.

In some implementations, end portions of helical groove 162 may have a geometry or surface characteristic such that the rotational and translational speed and momentum of cannula 28 is automatically dampened or braked as it approaches its end of travel along stylet 24. Such braking may reduce impact forces and noise that might otherwise occur when cannula 28 reaches its end of travel along stylet 24. Such impact forces and noise are often disconcerting to the patient. By reducing such impact forces and noise, biopsy device 120 may provide the patient with a less stressful and more tolerable experience.

In some implementations, selected end portions of helical groove 162 have a geometry and/or surface characteristic such that the rotational and translational speed and momentum of cannula 28 is automatically dampened or braked after the leading edge of cannula 28 has translated past the distal most axial end of slot 38, the end of slot 38 close to tip 36. This facilitates quick and abrupt severing of the tissue sample captured within slot 38 while, at the same time, reducing the impact noise and force following such tissue severing. In one implementation, location at which the geometry and/or surface characteristics of helical groove 162 change to initiate such braking is such that braking begins once the distal cutting-edge 50 of cannula 28 has moved at least 5 mm past the distal most and of slot 38. In other implementations, this breaking initiation location may vary depending upon the end of travel of cannula 28 relative to the distal end of slot 38. In other implementations, the rotational and translational speed of cannula 28 may have other speed adjustment profiles relative to the axial length of stylet 24.

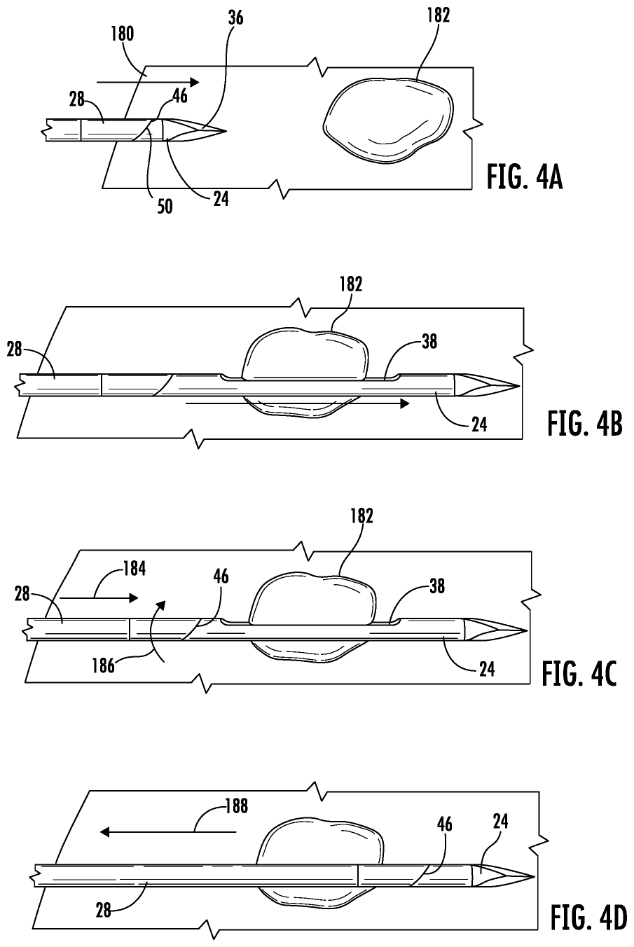

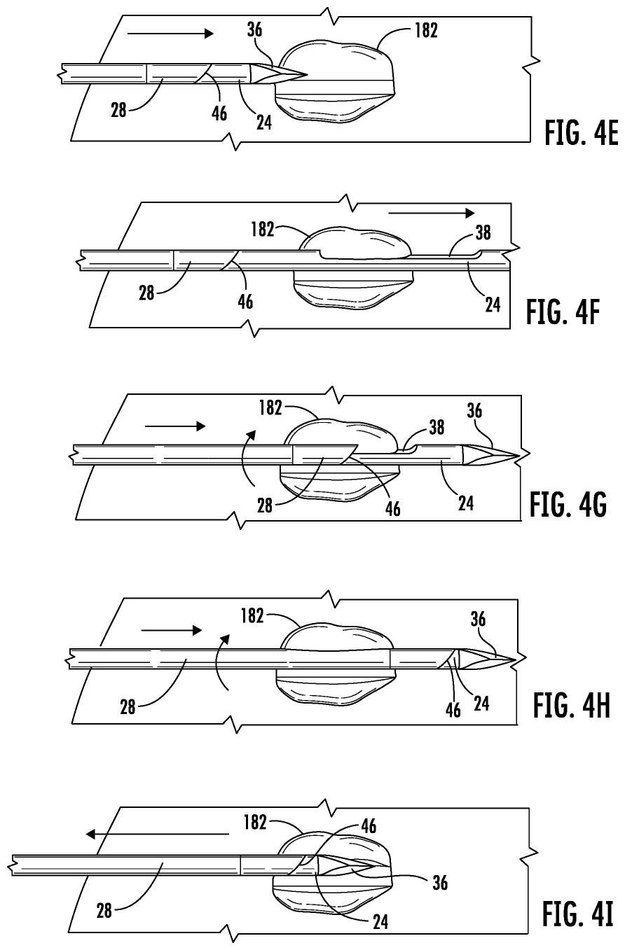

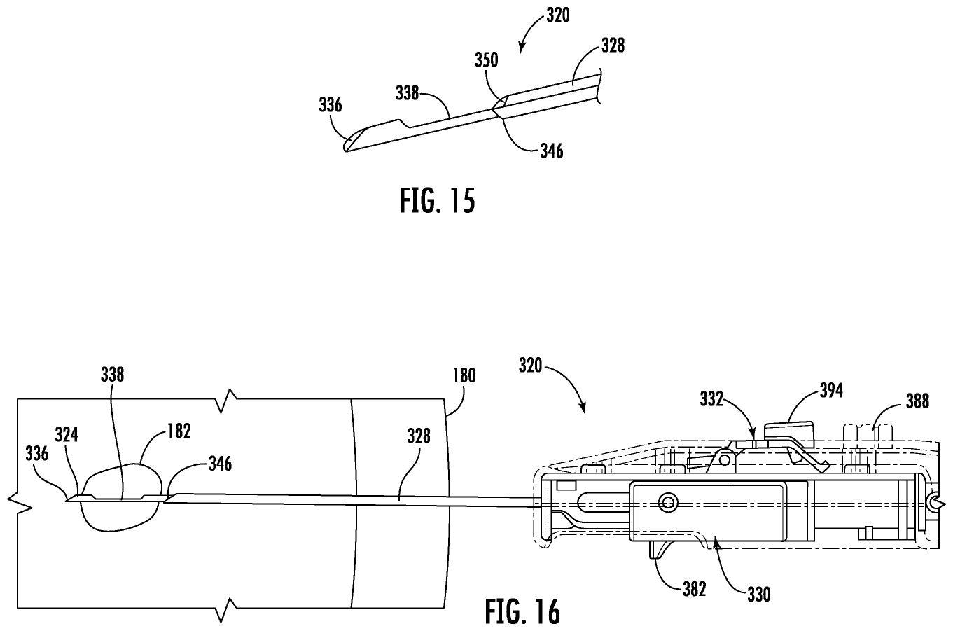

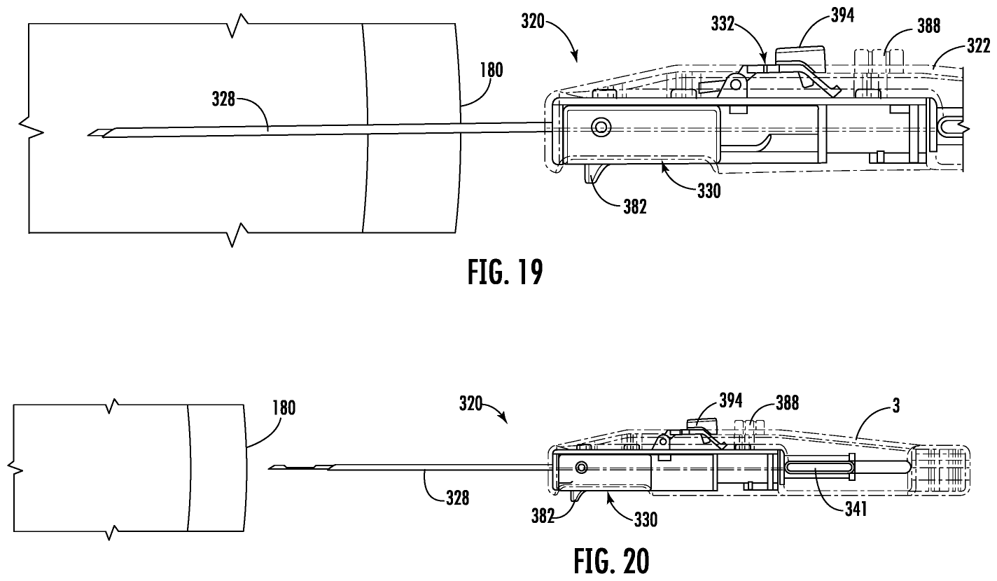

FIGS. 4A-41 illustrate one example for the acquisition of a biopsy sample by device 20 or device 120. As shown by FIG. 4A, stylet 24 and cannula 28 are inserted into biological tissue 180 towards tissue 182 of interest with slot 38 covered by cannula 28. As shown by FIG. 4B, stylet 24 is extended from cannula 28 to uncover slot 38 opposite to the tissue 182 of interest. The tissue 182 of interest, following initial compression, relaxes into slot 38.

As shown by FIG. 4C cannula 28 is linearly translated along the axial centerline of stylet 24 relative to stylet 24 as indicated by arrow 184. As indicated by arrow 186, cannula 28 is also rotated about the axial centerline of stylet 24. This process continues until the tip 46 of stylet 28 has been moved past slot 38. As cannula 28 moves across slot 38 during linear translation, cutting edge 50 is rotated to carry out a side cutting action of the tissue 182 of interest. This side cutting action results in a cleaner cut of the tissue 182 of interest, resulting in a larger mass or sample of the tissue 182 of interest being received within and captured within slot 38 as compared to cannula 28 simply being linearly translated along stylet 24 during the cutting of the tissue 182 of interest. As shown by FIG. 4D, once slot 38 has been covered by cannula 28, capturing and retaining the sample of tissue 182 within slot 38, stylet 24 and cannula 28 are both linearly retracted as indicated by arrow 188, withdrawing stylet 24 and cannula 28 from the biological tissue 180. During such retraction, slot 38 remains covered by cannula 28. Once withdrawn, cannula 28 is retracted and/or stylet 24 is extended to uncover slot 38 for the removal of the collected biological sample.

FIGS. 4E-41 illustrate the same process during the collection of a second biological sample from the tissue 182 of interest.

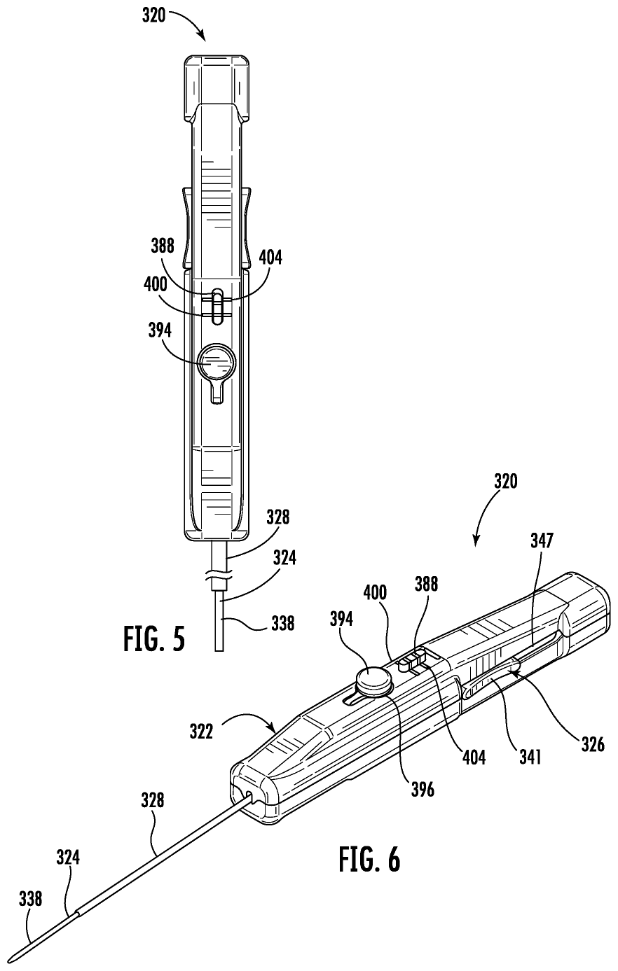

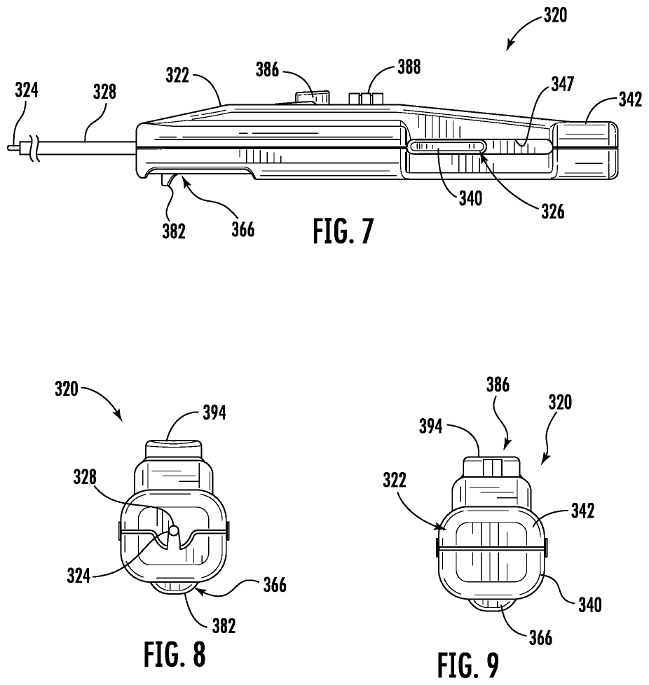

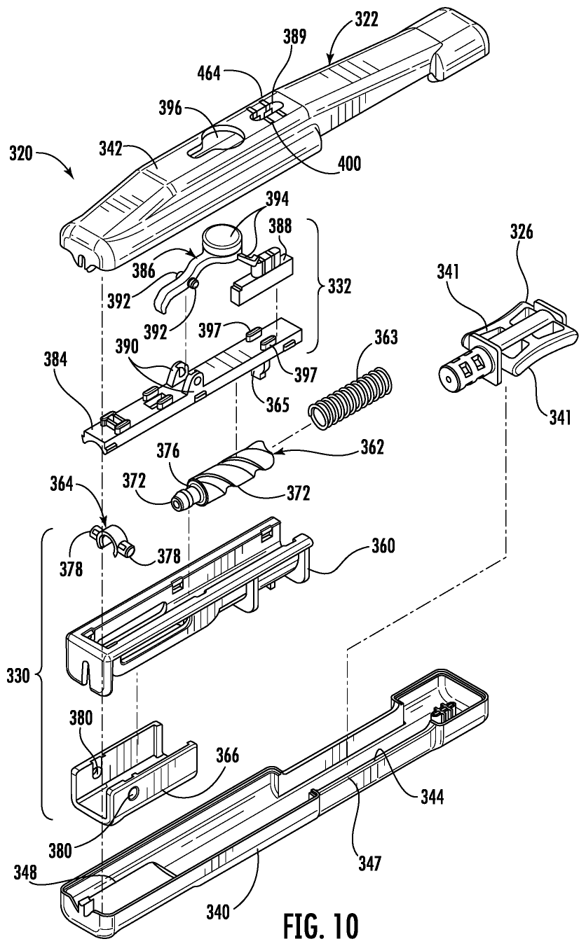

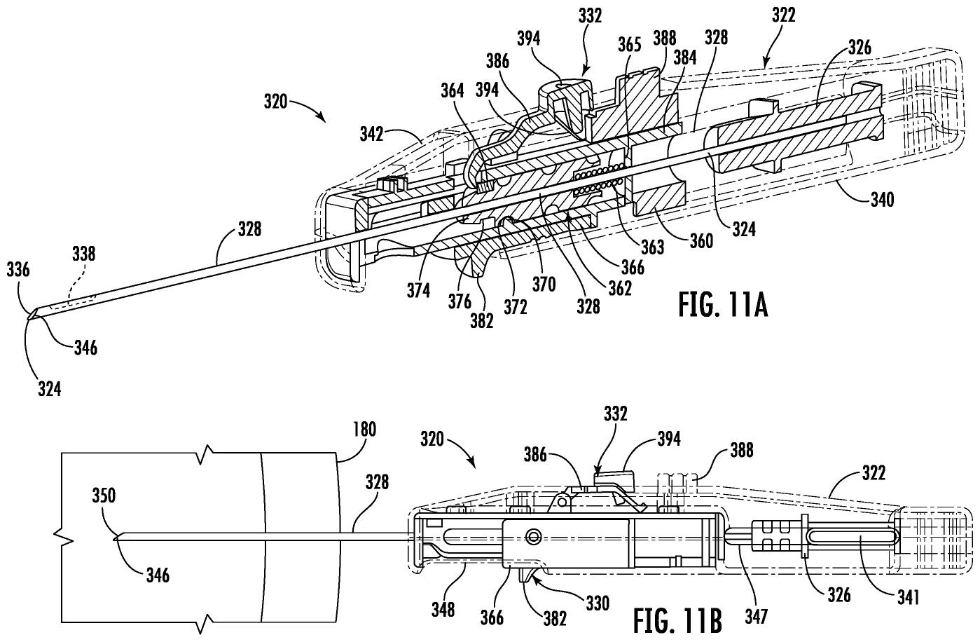

FIG. 5 is a top view of biopsy device 320. FIG. 6 is a top perspective view of biopsy device 320. FIG. 7 is a side view of biopsy device 320. FIG. 8 is a front view of biopsy device 320. FIG. 9 is a rear view of biopsy device 320. FIG. 10 is an exploded perspective view of biopsy device 320, omitting stylet 324 and cannula 328. FIG. 11A is a sectional view of biopsy device 320.

As shown by FIGS. 5-11A, biopsy device 320 comprises housing 322, stylet 324, stylet drive 326, cannula 328, cannula drive 330, and cannula lock 332. Housing 322 encloses the internal components of device 320. Housing 322 is formed from a lower shell 340 and an upper shell 342. Lower shell 340 and upper shell 342 cooperate to form an internal channel 344 in which stylet drive 324 moves and is guided. Lower shell 340 comprises an opening 347 through which stylet drive 326 projects for being manually engaged. Lower shell 340 further comprises opening 348 through which portions of cannula drive 330 project for being manually engaged.

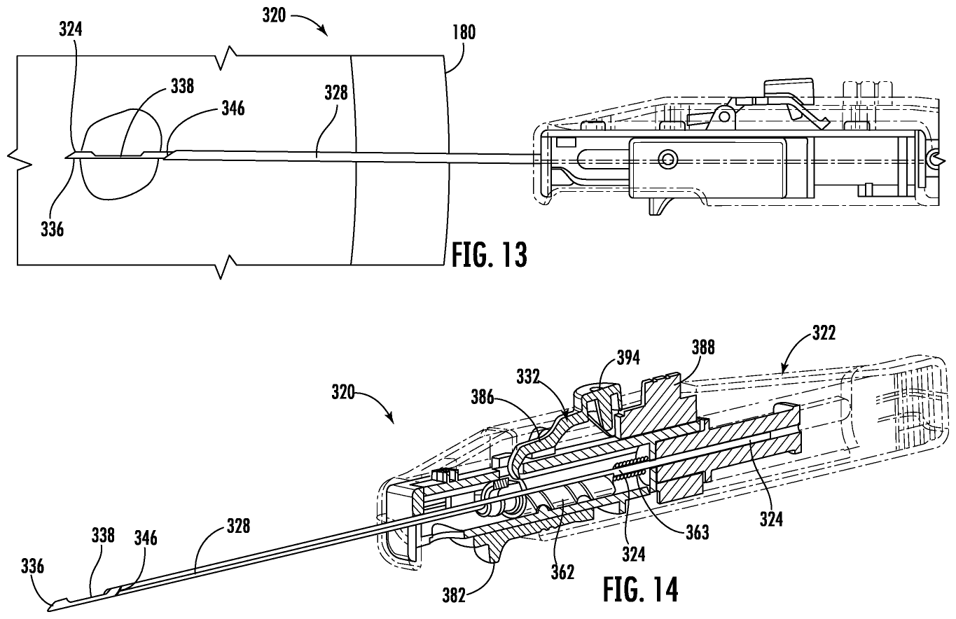

Stylet 324 is similar to stylet 24 described above. Stylet 324 comprises an outer slot 338 (shown in FIG. 13B) proximate the stylet tip 336. Stylet 324 has an end portion distant tip 336 that is fixedly connected to stylet drive 326 (shown in FIG. 11A). Stylet 324 has an outer slot 338 shown in an uncovered position or station FIG. 11A.

Stylet drive 326 comprise a member that slides within channel 344 and that has wing grips 341 that through opening 347. Wing grips 341 may be engaged by a person's hand to facilitate sliding of stylet drive 326 along channel opening 347 to extend and retract stylet 324.

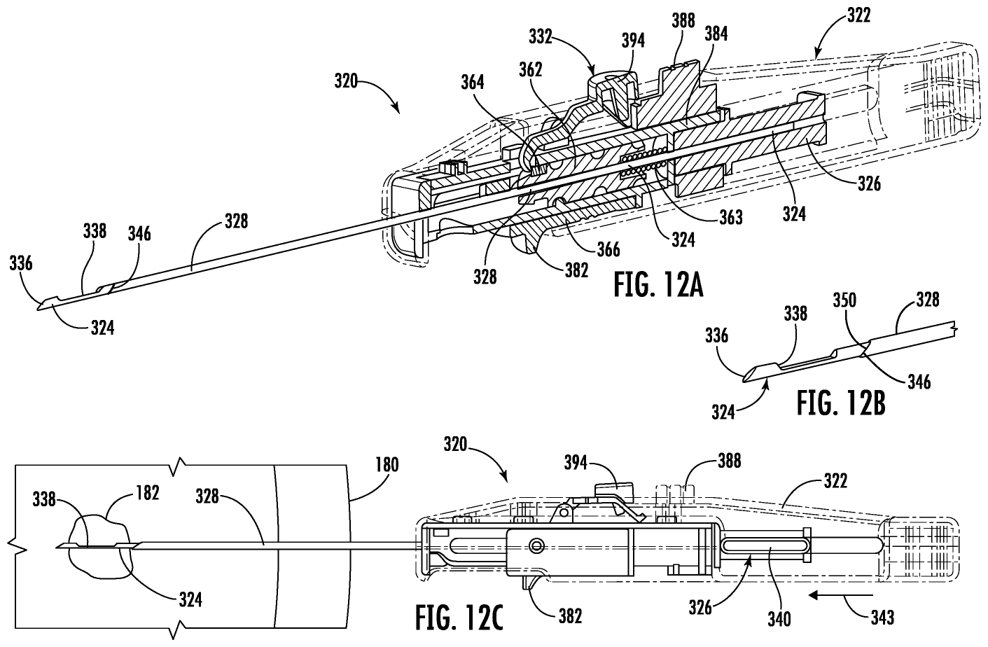

Cannula drive 330 comprises a mechanism to concurrently linearly translate and rotate cannula 328 relative to the internally received stylet 324. Cannula drive 330 comprises cradle 360, worm screw or gear 362, spring 363, collar/yoke 364 and slide handle 366. Cradle 360 is captured between shells 340 and 342. Cradle 360 receives and guides rotation of worm gear 362. As shown by FIG. 12A, cradle 360 comprises a tab 370 that projects into a helical thread or groove 372 of worm gear 362. Worm gear 362 further comprises a head 374 having a neck 376 about which yoke 364 mounts. Worm gear 362 is fixedly mounted to cannula 328 such that linear translation or rotation of worm gear 362 also linearly rotates and translates cannula 328. Spring 363 comprises a compression spring captured between worm screw or gear 362 and spring baffle 365 of platform 384. Yoke 364 comprises a pair of outwardly extending pins 378 that are mounted within apertures 380 of slide handle 366. Slide handle 366 projects through opening 348 and shell 340 and comprises a lower rib 382 (shown in FIG. 11A) for being manually gripped or pushed/pulled. Rearward sliding movement of handle 366 rearwardly translates worm gear 362 and cannula 328, compressing spring 363 against baffle 365 people loaded” cannula drive 330. Cannula drive 330 is retained in the loaded state by cannula drive lock 332 until released, resulting in spring 363 driving helical groove 372 and cannula 328 in a forward direction relative to and along stylet 324.

Cannula drive lock 332 locks cannula drive 330 in a loaded state to inhibit rotation or translation of cannula 328 as stylet 324 undergoes extension or retraction by stylet drive 326. Cannula drive lock 332 comprises platform 384, locking hook 386 and lock actuator 388. Platform 384 mounts within housing 322 on top of cradle 360. Platform 384 comprises a pair of ears 390 that receive pins 392 of locking of 386 to pivotably support locking hook 386 between a locking position (shown in FIG. 11A) and an unlocked position. Locking hook 386 projects through platform 384 and has a hooked end that engages yoke 364 to inhibit linear translation of yoke 364 and the resulting rotation and translation of worm gear 362 and cannula 328. Locking hook 386 comprises a pushbutton 394 that projects through opening 396 in shell/housing 322. Locking hook 386 further comprises a tether 398 connected to lock actuator 388. Lock actuator 388 comprises a bar that slides at top platform 384 and is guided by guide bars 397. Lock actuator 388 projects through an opening 389 formed in shell 342 of housing 322. Sliding movement of actuator 388 pivots hook 386 about pins 392 between the locked and unlocked positions. Hook 386 may be pivoted to the locking position by sliding actuator 388 forwardly. When slid to a fully forward position, actuator 388 is aligned with marking or indicia 400 indicating a “lock” state. In the locked state, actuator 388 and inhibits depression of pushbutton 394 and maintains hook 386 and engagement with collar 364, retaining biopsy device 320 in the loaded state or cocked state in which spring 363 remains compressed. Conversely, the sliding of actuator 388 rearwardly so as to be aligned with an indicia of “work” allows pushbutton 394 to be depressed, pivoting hook 386 to an unlocked position out of engagement with collar/yoke 364. The release of collar 364 in response to the depression of pushbutton 394 results in spring 363 urging worm gear 362 and cannula 368 forward along and relative to stylet 324. During such forward movement, cannula 368 also rotates as described above.

To reload device 320, a person may manually grip rib 382 and slide rib 382 rearwardly. Such sliding movement of slide handle 366 also linearly moves yoke 364. Linear translation of yoke 364 by handle 366 also results in linear movement or translation of worm gear 362 rearwardly, once again compressing spring 363. During linear movement of worm gear 362, helical groove 372 bears against tab 370 which causes rotation of worm gear 362 (and cannula 328) about the axial centerline of both cannula 328 and stylet 324. Worm gear 362 rotates relative to yoke 364 which is held against rotation by handle 366 and cradle 360. Once slid rearwardly to a sufficient degree, hook 386 reengaged his collar 364. To prevent inadvertent release of hook 386, actuator 388 may be slid forward to the “lock” state, temporarily retaining hook 386 in place by inhibiting pivoting of hook 386.

FIGS. 11B-20 illustrate the collection of a biopsy sample using biopsy device 320. FIGS. 11A and 11B illustrate biopsy device 320 during initial insertion of stylet 324 and cannula 328 into biological tissue 180, such as a breast. During such insertion, stylet 324 is sheathed within cannula 328 with cannula 328 extending over and covering slot 338. In one implementation, tip 336 of stylet 324 has a closed pointed end which extends beyond tip 346 of cannula 328 for penetrating biological tissue 180 during such insertion. As further shown by FIG. 11B, during such insertion, stylet drive 326 and cannula drive 330 are fully retracted with cannula drive lock 332 in a locked state.

As illustrated by FIGS. 12A, 12B, 12C and 13-16, once the tip of stylet 324 has is position proximate to the tissue 182 of interest for sampling, stylet actuator 326 is engaged via wing grips 341 and is slid forwardly in the direction indicated by arrow 343 within channel 344 of housing 322 (shown in FIG. 10) so as to further extend stylet 324, extending slot 338 out of cannula 328 to an uncovered position generally within the tissue 182 of interest. The tissue 182 of interest may expand or otherwise occupy the volume of slot 338.

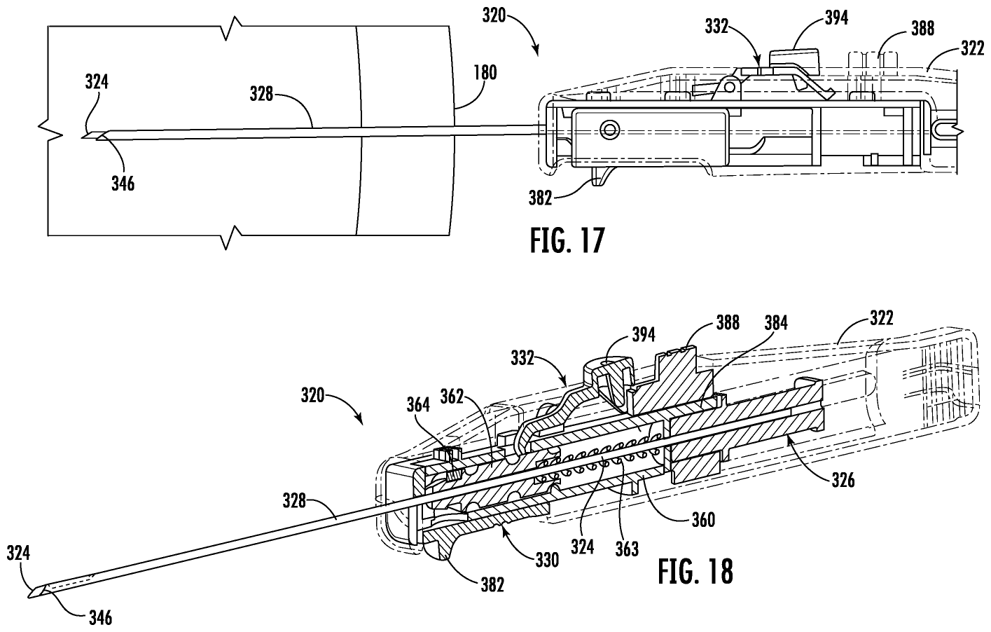

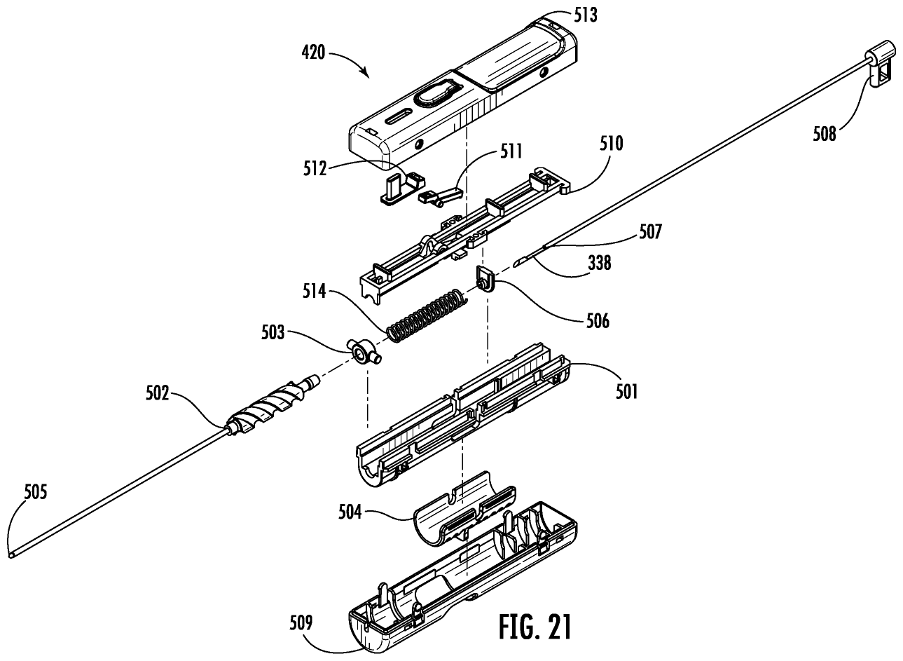

As illustrated by FIGS. 17-19, once the tissue 182 of interest has at least partially filled slot 338, cannula drive 330 is unlocked by the sliding of actuator 388 rearwardly and the depression of pushbutton 394, pivoting and releasing hook 386. Following the release of hook 386 from collar 364, compression spring 363 decompresses and transmits force to worm gear 362, pushing worm gear 362 and cannula 328 forward along and relative to the generally stationary stylet 324. During such forward movement of worm gear 362, tab 370 projecting from cradle 360, rides within the helical thread or groove 372 of worm gear 362 to concurrently rotate worm gear 362 and cannula 328. Such translation and concurrent rotation is continued until cannula 328 has been completely moved across slot 338 (as shown in FIGS. 17-19), severing the sample within slot 338 from the biological to show external to slot 338. Thereafter, as shown by FIG. 20, with the sample completely contained within slot 338, both stylet 324 and cannula 328 are withdrawn from biological tissue 180.

Following such withdrawal, rib 382 may be manually engaged to pull slight handle 366 rearwardly, retracting cannula 328 relative to stylet 124 so as to uncover slot 338, facilitating withdrawal of the obtained sample from slot 338. As described above, because cannula 328 is rotated as it is being manually moved across slot 338, cannula 328 provides a side cutting action. The side cutting action may achieve the retrieval of a larger sized sample from the single insertion. The larger sized biopsy sample may enhance analysis or reduce the number of tissue invasions carried out to obtain a sufficient quantity of biological tissue of interest for analysis.

FIG. 21 is an exploded perspective view of another example biopsy device 420. Biopsy device 420 is similar to biopsy device 320 described above except that biopsy 420 has a collar 503 is position rearwardly of worm gear 502 rather than forward of worm gear 502. In addition, the relationship of baffle 511 (corresponding to hook 386) and safety pin 512 (corresponding to actuator 388) is reversed with safety pin 512 forward of baffle 511, wherein forward movement of safety pin 512 releases baffle 511, allowing baffle 511 to be unhooked, allowing spring 514 to drive worm gear 502 and cannula or cutting needle 505.

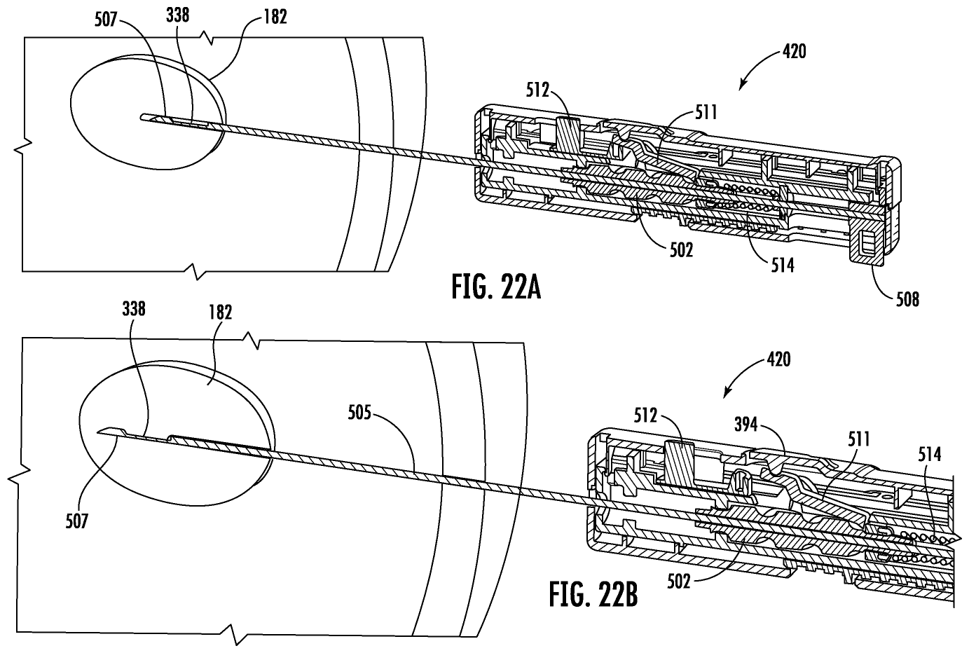

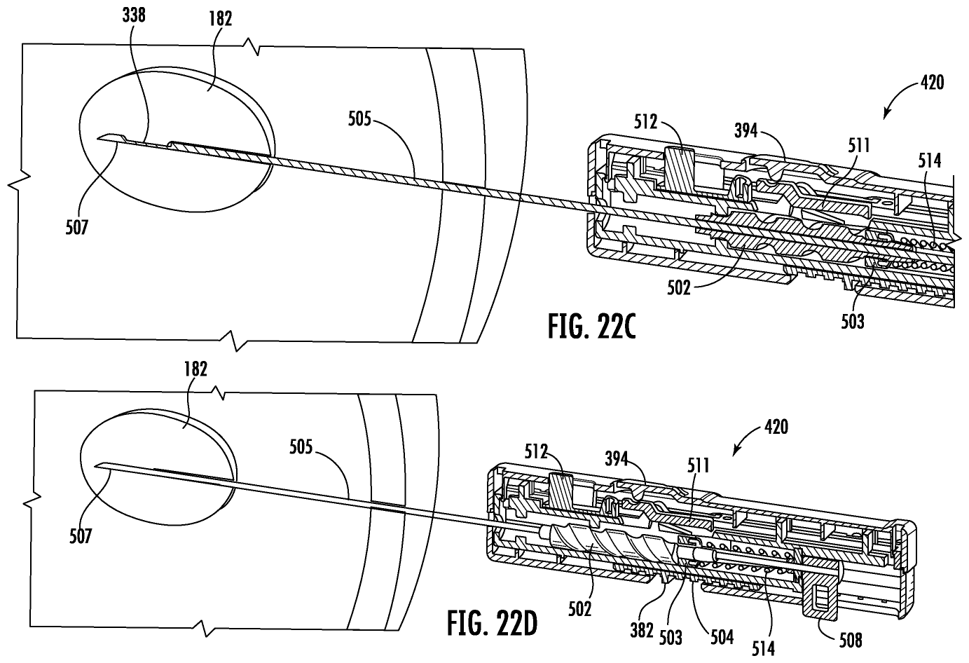

FIGS. 22A-22D illustrate use of biopsy device 420, taking a sample from tissue 182. FIG. 22A illustrates biopsy device 420 in a loaded state in which spring 514 is in a compressed state and in which the rotating base or worm gear 502 is loaded. FIG. 22B illustrates safety pin 512 being slid forward to an unlocked state which permits the depression of pushbutton 394 to pivot baffle 511 and unhook worm gear 502 or collar 503 (corresponding to collar 364). FIG. 22C illustrates biopsy device 420 following the depression and of pushbutton 394 to pivot baffle 511 and fire or release worm gear 502. FIG. 22D illustrates the extension of the compression spring 514, driving cannula 505 over and past the tissue receiving slot 338 of the stylet 507. Thereafter, worm gear 502 and cannula 505 may be reloaded by pulling back on handle 366 of sleeve 504, compressing spring 514 until baffle 511 may once again hooks into engagement with collar 503 or the end of worm gear 502. Stylet 507 may be moved relative to cannula 505 through actuation of stylet endcap 508 (corresponding to wing grips 341 of device 320).

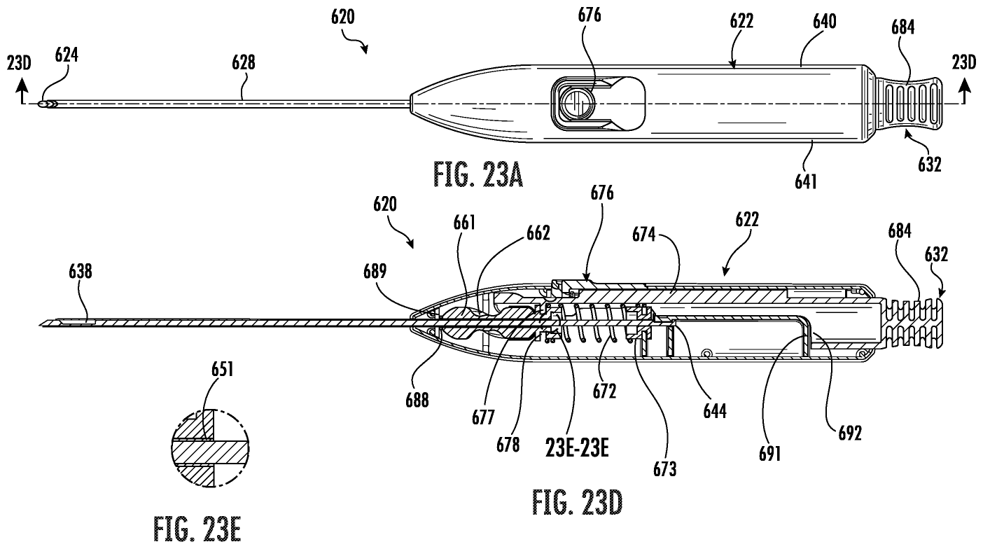

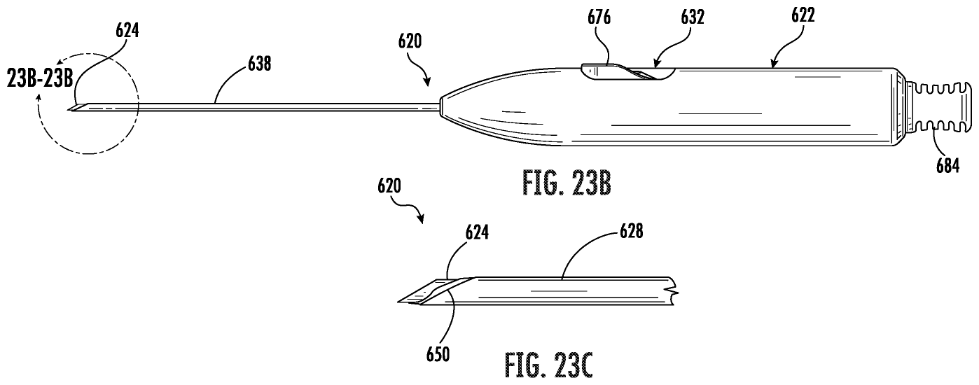

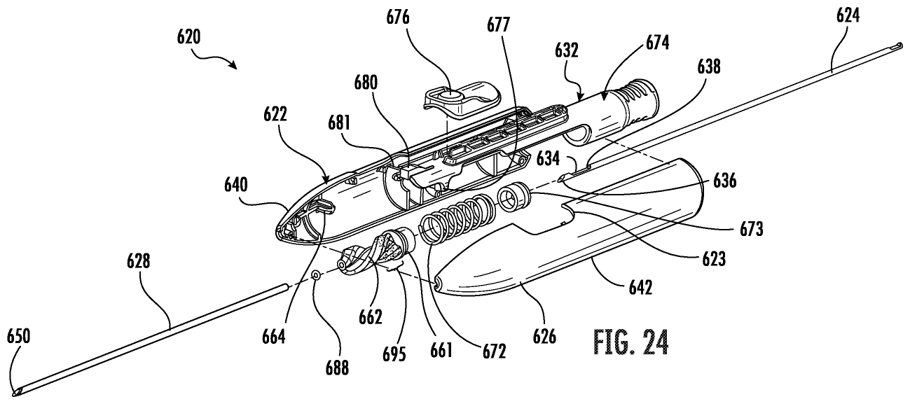

FIGS. 23A, 23B, 23C, 23D and 23E illustrate an example biopsy device 620. FIG. 24 is an exploded perspective view of biopsy device 620. As shown by FIG. 24, biopsy device 620 comprises housing 622, stylet 624 (inner needle), cannula 628 (outer needle) and cannula drive 632. Housing 622 guides and supports remaining components of biopsy device 620. In the example illustrated, housing 622 comprises base housing 640 and cover housing 642. Base housing 640 guides movement of cannula drive 632 and mates with cover housing 642 to enclose remaining components of biopsy device 620.

Stylet 624 is similar to stylets 24 and 324 described above. Stylet 624 comprises a slot 638 having a distal and 639 proximate a distal tip 636. As shown by FIG. 23C, stylet 624 has a proximal and 644 affixed relative to housing 622. Stylet 624 serves as a guide for sliding and rotational movement of cannula 628 by cannula drive 632.

Cannula 628 is similar to cannulas 28 and 328 described above. Cannula 628 extends over in about stylet 624. Cannula 628 has a cutting-edge 650 similar to cutting edge 50 described above. As shown by FIG. 23D, cannula 628 has a proximal end 651 that affixed relative to worm gear 661 of cannula drive 632. In one implementation, proximal end 651 is press-fit into worm gear 661. In some implementations, welding, adhesive or other joining techniques may be employed to fix cannula 628 relative to worm gear 661.

Cannula drive 632 concurrently rotates and translates cannula 628 overran along stylet 624. Cannula drive 632 comprises worm gear 661, tab 664, spring 672, spring insert 673, spring carrier 674 and actuation button 676. Worm gear 661 has a helical groove 662 which receives tab 664. Tab 664 comprises a projection extending from housing 622. Translation of worm gear 663 along the axis of stylet 624 results concurrent rotation of worm gear 661. As noted above, worm gear 661 is affixed to cannula 628 such that rotation and translation of worm gear 662 as a result of its interaction with tab 664 also results in rotation and translation of cannula 628 along and relative to stylet 624.

Spring 672 comprises a compression spring captured between worm gear 661 and spring insert 673. As shown by FIG. 23C, spring insert 673 is affixed to housing 622 and retains one end of spring 672. In some implementations, spring insert 673 may be omitted, wherein spring 672 is captured and abuts the internally projecting walls of housing 622.

Spring carrier 674 is slidably disposed within housing 622. As shown by FIG. 23C, spring carrier 674 has a downwardly projecting catch 677 which is received within a circumferential groove 678 formed in worm gear 661. As a result, worm gear 661 is rotatable about the axis of stylet 624 relative to carrier 674, but is axially movable or translatable with spring carrier 674. Spring carrier 674 further comprising an upwardly extending or projecting catch 680 configured to engage and catch upon shoulder 681 of housing 622 when spring carrier 674 is pulled back or retracted to a predefined spring loading state or position. Catch 680 is resiliently biased upwards into engagement with shoulder 681 by spring 672. Spring carrier 674 further comprises a handle or plunger 684 at its proximal end. Plunger 684 projects from housing 622, facilitating manual movement of spring carrier 674 and loading of spring 672.

Actuation button 676 is captured within housing 622, projecting through opening 623 for manual depressment. Depressment of actuation button 676 depresses catch 680 of spring carrier 674 out of engagement with shoulder 681 of housing 622, permitting spring carrier 674 to move within and relative to housing 622 under the force of spring 672. As a result, depressment of button 676 results in firing of biopsy device 620 with the rotation and translation of both worm gear 661 and cannula 628 along stylet 624.

The rotation and linear translation of cannula 628 is limited by an O-ring 688 which extends about cannula 628, between a stop surface 689 provided by housing 622 and a distal end of worm gear 661. O-ring 688 serves as a bumper to cushion the end of travel of worm gear 661 towards the distal end of stylet 624. In some implementations, O-ring 688 may be omitted. In some implementations, a bumper pad formed from a rubber or elastomeric polymer may be provided between housing 622 and worm gear 661, being formed on the distal tip of worm gear 661 and/or being formed upon the opposing surface of housing 622, providing surface 689. As shown by FIG. 23C in some implementations, the translational movement of cannula 628, worm gear 661 and spring carrier 674 may further (or alternatively) be limited by the engagement of housing shoulder 691 and a corresponding shoulder surface 692 and spring carrier 674. In such implementations, a bumper pad may be provided on either or both of such surfaces 691 and 692

In some implementations, different axial segments of helical groove 162 have geometries (a tighter or closer fit with respect to tab 164) or surface characteristics (a rougher surface or a coating or layer of material with a higher coefficient of friction with respect to the material of tab 164) that result in different degrees of friction and different degrees of resistance with respect to relative movement between tab 664 and helical groove 662. As a result, the speed at which cannula 628 rotates and moves about stylet 624 may be adjusted automatically based upon the actual positioning of cannula 628 relative to stylet 624. Cannula 628 may rotate and translate at a first nonzero speed across a first portion of stylet 624 and may rotate and translate at a second nonzero speed across a second different portion of stylet 624.

As shown by FIG. 24, in some implementations, end portions 695 of helical groove 662 may have a geometry or surface characteristic (represented by stippling) such that the rotational and translational speed and momentum of cannula 628 is automatically dampened or braked as it approaches its end of travel along stylet 624. For example, end portion 695 is coated with a higher friction material (represented by stippling), such as an elastomeric polymer or rubber material and/or remaining portions of worm gear 661, other than in portion 695, may be coated with a lower friction material such as polytetrafluoroethylene. In some implementations, end portion 695 is additionally or alternatively be provided with a textured surface (represented by stippling), wherein other axial segments or portions of helical groove 662 are smoother. In some implementations, end portion 695 is slightly enlarged (represented by stippling) relative to remaining portions or axial segments of helical groove 662 so as to extend into closer, higher friction abutment with the tip and/or sides of tab 664. Such braking may reduce impact forces and noise that might otherwise occur when cannula 628 reaches its end of travel along stylus 624. Such impact forces and noise are often disconcerting to the patient. By reducing such impact forces and noise, biopsy device 620 may provide the patient with a less stressful and more tolerable experience.

In some implementations, selected end portions 695 of helical groove 662 have a geometry and/or surface characteristic (represented by stippling) such that the rotational and translational speed and momentum of cannula 628 is automatically dampened or braked after the leading edge of cannula 628 has translated past the distal most axial end of slot 638, the end of slot 638 close to tip 636. This facilitates quick and abrupt severing of the tissue sample captured within slot 638 while, at the same time, reducing the impact noise and force following such tissue severing. In one implementation, location at which the geometry and/or surface characteristics of helical groove 662 change to initiate such braking is such that braking begins once the distal cutting-edge 650 of cannula 628 has moved at least 5 mm past the distal most end of slot 638. In other implementations, this braking initiation location may vary depending upon the end of travel of cannula 628 relative to the distal end of slot 638. In other implementations, the rotational and translational speed of cannula 628 may have other speed adjustment profiles relative to the axial length of stylet 624.

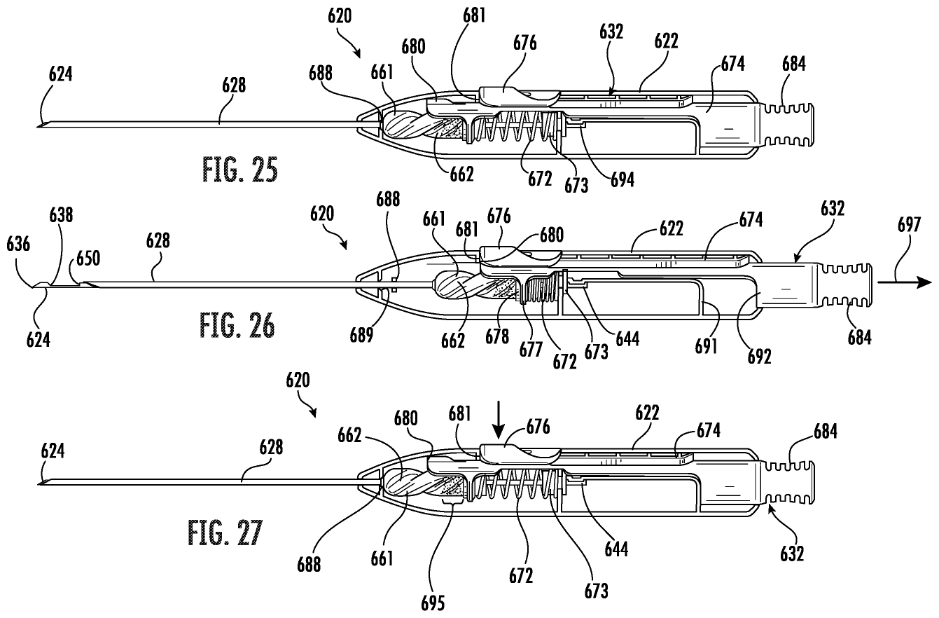

FIGS. 25-27 illustrate use of biopsy device 620. FIG. 25 illustrates biopsy device 620 in an unloaded state. FIG. 26 illustrates a loading of biopsy device 26 through the manual pulling of plunger 684 in the direction indicated by arrow 697. Such retraction occurs until 680 has been moved back beyond shoulder 681. At such time, spring 672 resilient urges catch 680 upward into locking engagement with shoulder 681. In such a positioning, cannula 628 is retracted to expose slot 638.

FIG. 27 illustrates a firing of biopsy device 620. Firing is initiated through the depression of button 676 which results in catch 680 being depressed out of engagement with shoulder 681. As a result, spring 672 decompresses, urging worm gear 661 and cannula 628 axially along stylet 624. During such translation, tab 664 interacts with the helical groove 662 of worm gear 6612 concurrently cause worm gear 661 and cannula 628 to rotate about stylet 624. Such translation and rotation continues until worm gear 661 engages O-ring 688. As discussed above, in the example illustrated, the higher friction between segment 695 of worm gear 661 and tab 664 (shown in FIG. 24) results in the speed at which worm gear 661 rotate and translate being braked as it approaches tip 636 of stylet 624. In the example illustrated, tab 664 does not reach and interact with segment 695 of helical groove 662 until cutting-edge 650 of cannula 628 has moved axially past slot 638 thought to extend between slot 638 and tip 636. As noted above, in the example illustrated, segment 695 of helical groove 662 may have a geometry, surface texture roughness or material coating that is different than the remaining more distal portions of helical groove 662 to provide segment 695 with enhanced friction relative to the remaining distal portions of helical groove 662. In other implementations, helical groove 662 may have a uniform level of friction along its length with respect to tab 664.

Although the present disclosure has been described with reference to example implementations, workers skilled in the art will recognize that changes may be made in form and detail without departing from the scope of the claimed subject matter. For example, although different example implementations may have been described as including features providing benefits, it is contemplated that the described features may be interchanged with one another or alternatively be combined with one another in the described example implementations or in other alternative implementations. Because the technology of the present disclosure is relatively complex, not all changes in the technology are foreseeable. The present disclosure described with reference to the example implementations and set forth in the following claims is manifestly intended to be as broad as possible. For example, unless specifically otherwise noted, the claims reciting a single particular element also encompass a plurality of such particular elements. The terms “first”, “second”, “third” and so on in the claims merely distinguish different elements and, unless otherwise stated, are not to be specifically associated with a particular order or particular numbering of elements in the disclosure.

What is claimed is:

1. A biopsy device comprising:
a stylet having a stylet tip and an outer slot for receiving a biopsy sample;
a cannula extending about and receiving the stylet, the cannula having a cannula tip with a cutting edge facing an axial centerline of the cannula; and
a cannula drive coupled to the cannula to translate and rotate the cannula along and about the axial centerline of the cannula from a first position in which the outer slot is uncovered and a second position in which the cannula extends over and covers the outer slot.

2. The biopsy device of claim 1, wherein the cannula drive concurrently translates and rotates the cannula along and about the axial centerline of the cannula.

3. The biopsy device of claim 1, wherein the cutting edge extends in a plane 45° to the axial centerline of the cannula.

4. The biopsy device of claim 1, wherein the cutting edge extends in a plane less than 45° to the axial centerline of the cannula.

5. The biopsy device of claim 1, wherein the cutting edge comprise different portions about the axial centerline, at least one of the different portions extending in a plane less than 45° to the axis centerline of the cannula.

6. The biopsy device of claim 1 further comprising a lock to inhibit movement of the cannula drive.

7. The biopsy device of claim 1 further comprising a housing about the cannula, wherein the cannula drive comprises a helical groove and a tab projecting into the helical groove, wherein one of the helical groove and the tab is connected to the cannula, wherein the other of the helical groove and the tab is connected to the housing.

8. The biopsy device of claim 7, wherein the cannula drive further comprises a handle connected to said one of the helical groove and the tab, the handle being movable in a direction along an axial centerline of the stylet to rotate the cannula while linearly translating the cannula, relative to the stylet, along the axial centerline of the stylet.

9. The biopsy device of claim 8, wherein the handle is connected to the helical groove.

10. The biopsy device of claim 7, wherein the cannula drive further comprises a spring connected to said one of the helical groove and the tab to urge said one of the helical groove and the tab in a direction along an axial centerline of the stylet to rotate the cannula while linearly translating the cannula, relative to the stylet, along the axial centerline of the stylet.

11. The biopsy device of claim 10, wherein the cannula drive further comprises a spring retainer movable between charged state in which the spring retainer retains the spring in a loaded state and a discharged state that allows the spring to urge said one of the helical groove and the tab in a direction along an axial centerline of the stylet to rotate the cannula while linearly translating the cannula, relative to the stylet, along the axial centerline of the stylet.

12. The biopsy device of claim 10, wherein, wherein the helical groove has a first axial segment having a first degree of friction with the tab and a second axial segment having a second degree of friction with the tab, the first degree of friction being different than the second degree of friction.

13. The biopsy device of claim 12, wherein the helical groove has varying degrees of friction with respect to the tab along its axial length so as to automatically initiate braking of rotation and translation of the cannula upon the cannula being located beyond the outer slot.

14. The biopsy device of claim 12, wherein the first axial segment has a first roughness, a first surface material or a first fit with respect to the tab and wherein the second axial segment has a second roughness, a second surface material or a second fit with respect to the tab, the second roughness, the second surface material and the second fit with respect to the tab being different than the first roughness, the first surface material and the first fit with respect to the tab, respectively.

15. The biopsy device of claim 10 further comprising a manually actuatable plunger to load the spring.

16. The biopsy device of claim 1 further comprising a stylet drive coupled to the stylet to linearly translate the stylet relative to the cannula between a retracted position in which the outer slot is covered by the cannula and an extended position in which the outer slot is uncovered, the stylet drive being operable independent of the cannula drive.

17. A method comprising:
positioning an outer slot of a stylet adjacent biological tissue; and
concurrently rotating and translating a cannula along the stylet by linearly driving a helical groove connected to the cannula, the cannula having a tip with a cutting edge facing an axial centerline of the cannula.

18. The method of claim 17 further comprising locking the cannula against rotation.

19. The method of claim 17 further comprising:
loading a spring; and
releasing the spring to apply force to the cannula to linearly translate and rotate the cannula.

20. The method of claim 17 further comprising sliding a handle along an axial centerline of the stylet to linearly translate and rotate the cannula relative to and along the stylet.
